SEQUENCE LISTING (20 sequences)

SEQ ID NO: 1 - protein, 13 aa, Artificial
  MISC_FEATURE: peptide sequence antigen for antibodies against complementaryfactor C3a
KCCEDGMRQNPMR

SEQ ID NO: 2 - protein, 13 aa, Artificial
  MISC_FEATURE: peptide sequence antigen for antibodies against complementaryfactor C3a
RFSCQRRTRFISL

SEQ ID NO: 3 - protein, 12 aa, Artificial
  MISC_FEATURE: peptide sequence antigen for antibodies against complementaryfactor C3a
ITELRRQHARAS

SEQ ID NO: 4 - protein, 14 aa, Artificial
  MISC_FEATURE: peptide sequence antigen for antibodies against complementaryfactor C5a
QADYKDDDDKLPAE

SEQ ID NO: 5 - protein, 14 aa, Artificial
  MISC_FEATURE: peptide sequence antigen for antibodies against complementaryfactor C5a
DDKLPAEGLDIENS

SEQ ID NO: 6 - protein, 14 aa, Artificial
  MISC_FEATURE: peptide sequence antigen for antibodies against IL1a/B
NCYSENEEDSSSID

SEQ ID NO: 7 - protein, 13 aa, Artificial
  MISC_FEATURE: peptide sequence antigen for antibodies against IL1a/B
GAYKSSKDDAKIT

SEQ ID NO: 8 - protein, 12 aa, Artificial
  MISC_FEATURE: peptide sequence antigen for antibodies against IL1a/B
WETHGTKNYFTS

SEQ ID NO: 9 - protein, 14 aa, Artificial
  MISC_FEATURE: peptide sequence antigen for antibodies against IL1a/B
RISDHHYSKGFRQA

SEQ ID NO: 10 - protein, 13 aa, Artificial
  MISC_FEATURE: peptide sequence antigen for antibodies against IL1a/B
VQGEESNDKIPVA

SEQ ID NO: 11 - protein, 17 aa, Artificial
  MISC_FEATURE: peptide sequence antigen for antibodies against IL1a/B
ESVDPKNYPKKKMEKRF

SEQ ID NO: 12 - protein, 17 aa, Artificial
  MISC_FEATURE: peptide sequence antigen for antibodies against IL6
APHRQPLTSSERIDKQI

SEQ ID NO: 13 - protein, 13 aa, Artificial
  MISC_FEATURE: peptide sequence antigen for antibodies against IL6
QNRFESSEEQARA

SEQ ID NO: 14 - protein, 12 aa, Artificial
  MISC_FEATURE: peptide sequence antigen for antibodies against IL6
AITTPDPTTNAS

SEQ ID NO: 15 - protein, 13 aa, Artificial
  MISC_FEATURE: peptide sequence antigen for antibodies against IL10
SPGQGTQSENSCT

SEQ ID NO: 16 - protein, 14 aa, Artificial
  MISC_FEATURE: peptide sequence antigen for antibodies against IL10
QMKDQLDNLLLKES

SEQ ID NO: 17 - protein, 13 aa, Artificial
  MISC_FEATURE: peptide sequence antigen for antibodies against IL10
MPQAENQDPDIKA

SEQ ID NO: 18 - protein, 12 aa, Artificial
  MISC_FEATURE: peptide sequence antigen for antibodies against IL10
LPCENKSKAVEQ

SEQ ID NO: 19 - protein, 14 aa, Artificial
  MISC_FEATURE: peptide sequence antigen for antibodies against TNFa
VRSSSRTPSDKPVA

SEQ ID NO: 20 - protein, 17 aa, Artificial
  MISC_FEATURE: peptide sequence antigen for antibodies against TNFa
KSPCQRETPEGAEAKPW

(19) United States
(12) Patent Application Publication
(10) Pub. No.: US 2008/0255344 A1
(43) Pub. Date: Oct. 16, 2008

(54) Immuno-Adsorbers For Treatment of Inflammations

(76) Inventor: Hans-Werner Heinrich, Greifswald (DE)

Correspondence Address: Buchanan Ingersoll & Rooney, One Oxford Centre, 301 Grant Street, 20th Floor, Pittsburgh, PA, 15219, US

(21) Appl. No.: 11/665,945

(22) PCT Filed: Oct. 7, 2005
(86) PCT No.: PCT/DE2005/001797

§ 371 Date: Apr. 8, 2008

(30) Foreign Application Priority Data
Oct. 20, 2004 (DE) 10 2004 051 216.7
Jan. 20, 2005 (DE) 10 2005 003 190.0

(51) Int. Cl. C07K 19/00

(52) U.S. Cl. 530/391.1

(57) ABSTRACT

The invention relates to an immunological adsorber for treating inflammations, in particular for eliminating complement factors, and, optionally, additional sepsis-mediators, such as, TNF and interleukins from body fluids. The invention also relates to a method for the production and use of said immunological adsorber. The aim of the invention is to develop a modular immunoadsorption system, in particular for extracorporal detoxification, which enables plasma and tissue levels, which are specific to the patient, to be reduced. The inventive immunological adsorber consists of organic or synthetic polymer support materials whereon polyclonal and monoclonal antibodies are connected which are oriented counter to the complement factor C3a and/or C5a. In a preferred embodiment, other antibodies which are oriented counter to the additional inflammation mediators, are connected to the support. In a particularly advantageous embodiment, the invention relates to an immunoadsorber provided with antibodies against C5a and IL6.

The invention relates to immunological adsorbers for the treatment of inflammation diseases, in particular for the removal of complementary factors and also, if applicable, interleukins and TNF from body fluids, methods for their manufacture and their use.

An immuno-adsorber for sepsis therapy is already known from WO 00/58005. It is characterised by carrier materials of organic or synthetic polymers, to which both poly- and also monoclonal antibodies aimed against the complementary factors C3a and/or C5a as well as antibodies aimed against lipopolysaccharides (LPS) are bound. In the preferred embodiment, antibodies aimed against further sepsis mediators are also bound to the carriers.

It is used for the removal of complementary factors and lipopolysaccharides (LPS) and also, if applicable, for the removal of further sepsis mediators and also of TNF and interleukins from body fluids. For example, it can be used for the plasma pheresis of patients with sepsis in this context.

The disadvantage of this adsorber system is that its application is limited to the “sepsis” inflammation of the entire body caused by gram negative bacteria. The production of poly-specific antibodies against the various lipopolysaccharides (LPS) is time-consuming and expensive in addition. Treatment of patients suffering from a sepsis caused by gram negative bacteria (e.g. Escherichia coli) with this adsorber produces good results. But this LPS adsorber is at least not necessary, but in any case too expensive for the therapy of a sepsis caused by other reasons. The aforementioned adsorber has its field of application in sepsis treatment as a result of its nature. It is not suited for other diseases connected with inflammations.

The invention is based on the task of developing an immuno-adsorption system with a modular construction, in particular for extra-corporal detoxification, making it possible to reduce the plasma and tissue level specific to the patient and not manifesting the aforementioned deficits.

This task is solved by the main claim, the sub-claims being preferential variants.

According to Claim 1, the immunological adsorber comprises organic and/or synthetic polymeric carrier materials, to which both poly- and/or monoclonal antibodies aimed against the complementary factors C3a and/or C5a are bound. In a preferential embodiment, antibodies aimed against other inflammation mediators are also bound to the carriers.

A particularly beneficial preferential embodiment of the invention is an immuno-adsorber with antibodies against C5a and IL6. Removal of C5a is important as this is the most effective inflammatory factor from the complementary cascade, and IL6, because this interleukin takes on a central function in the pro-inflammation regulation.

The antibodies used are preferably polyclonal antibodies, aviary antibodies of the type IgY being particular preferred. The antibodies against sepsis mediators are contained according to the condition of the dysregulation.

In accordance with the invention, it is a question of antibodies aimed against TNF, IL1, IL6, IL8 and/or IL10.

Preferred antibodies against the complementary factor C3a manifest specific activity against at least one of the following peptide sequences:

| | |
|---|---|
| NH₂-KCCEDGMRQNPMR-COOH | (SEQ ID NO 1) |
| NH₂-RFSCQRRTRFISL-COOH | (SEQ ID NO 2) |
| NH₂-ITELRRQHARAS-COOH | (SEQ ID NO 3) |

Preferred antibodies against the complementary factor C5a manifest specific activity against at least one of the following peptide sequences:

| | |
|---|---|
| NH₂-QADYKDDDDKLPAE-COOH | (SEQ ID NO 4) |
| NH₂-DDKLPAEGLDIENS-COOH | (SEQ ID NO 5) |

Preferred antibodies against IL1α/β manifest specific activity against at least one of the following peptide sequences:

| | |
|---|---|
| NH₂-NCYSENEEDSSSID-COOH | (SEQ ID NO 6) |
| NH₂-GAYKSSKDDAKIT-COOH | (SEQ ID NO 7) |
| NH₂-WETHGTKNYFTS-COOH | (SEQ ID NO 8) |
| NH₂-RISDHHYSKGFRQA-COOH | (SEQ ID NO 9) |
| NH₂-VQGEESNDKIPVA-COOH | (SEQ ID NO 10) |
| NH₂-ESVDPKNYPKKKMEKRF-COOH | (SEQ ID NO 11) |

Preferred antibodies against IL6 manifest specific activity against at least one of the following peptide sequences:

| | |
|---|---|
| NH₂-APHRQPLTSSERIDKQI-COOH | (SEQ ID NO 12) |
| NH₂-QNRFESSEEQARA-COOH | (SEQ ID NO 13) |
| NH₂-AITTPDPTTNAS-COOH | (SEQ ID NO 14) |

Preferred antibodies against IL10 manifest specific activity against at least one of the following peptide sequences:

| | |
|---|---|
| NH₂-SPGQGTQSENSCT-COOH | (SEQ ID NO 15) |
| NH₂-QMKDQLDNLLLKES-COOH | (SEQ ID NO 16) |
| NH₂-MPQAENQDPDIKA-COOH | (SEQ ID NO 17) |
| NH₂-LPCENKSKAVEQ-COOH | (SEQ ID NO 18) |

Preferred antibodies against TNFα manifest specific activity against at least one of the following peptide sequences:

| | |
|---|---|
| NH₂-VRSSSRTPSDKPVA-COOH | (SEQ ID NO 19) |
| NH₂-KSPCQRETPEGAEAKPW-COOH | (SEQ ID NO 20) |

The immuno-adsorber pursuant to the invention manifests membranes or particles of organic or synthetic polymers which are customary per se as carrier materials, e.g. made of polystyrols, carbohydrates such as cellulose or agarose derivatives, or of acrylates, the specific antibodies being bound to them covalently or fixed to them via spacers or linkers.

The immuno-adsorbers according to the invention are produced by methods known per se by the antibodies aimed against C3a and/or C5a and also, if applicable, against further sepsis mediators being coupled covalently or adsorptively to the carrier materials made of organic or synthetic polymers.

The specific antibodies are produced by immunisation known per se, preferably of small mammals such as mice, rats or rabbits, or of birds, e.g. chickens, with the corresponding antigens.

The object of the invention is also the use of the immuno-adsorbers in devices for the removal of complementary factors and, if applicable, of further mediators from body fluids, such as blood plasma, as a function of the specific situation of the patient.

The immuno-adsorbers are preferably used in sepsis therapy for plasma pheresis in patients with sepsis or a septic shock.

Although antibodies which can be coupled to the various carriers according to known methods are available for most substances, aviary antibodies are preferably used, as they do not activate the complementary system, unlike mammal antibodies. As the activating properties are coupled to the Fc part of the mammal antibodies, the Fab fragment split off with papain can principally also be used.

According to the current state of knowledge, immobilised aviary antibodies have no kind of unspecific effects on the human defence system. Birds, preferably chickens, are immunised with customary methods with or without use of adjuvants. The specific immunoglobulins are excreted in the egg yolk and can be isolated from this with customary methods. They are covalently bound to micro-particles or membranes via the Fc part with known methods.

The immuno-adsorption system for extra-corporal detoxification according to the invention provides a selective system which can be used specifically for certain patients and through which dysregulations of the immune system can be remedied for the first time.

The invention is to be explained in more detail below with examples.

### EXAMPLES OF EMBODIMENTS

### Example 1

### a) Activation of a Carrier

The IgY cleansed according to example 3 are covalently bound to a suitable carrier. For this, for example, sepharose can be activated as described below (H.-F. Boeden, W. Büttner, C. Rupprich, D. Büttner, S. Heinrich, M. Becker, M. Holtzhauer (1992), Makromol. Chem. 193, 865-887):

The agarose carrier is transferred step by step, i.e. by an amount of acetone increased by 20% each step. At the end, the carrier is left to stand over night in a quintuple bed volume with water-free acetone in a closed vessel and washed again with 5 to 10 vol. water-free acetone and briefly drained on a G2 fritte. 400 mg N-(chlorocarbonyloxy)-5-norbornen-2,3-dicarboximide (CICOONB) in 10 ml of water-free acetone p.a. are added to 10 ml of sedimented carrier. Within 15 minutes, a solution of 280 μl triethylamine and 20 mg 4-dimethlyaminopyridin (DMAP) is added to 5 ml of dry acetone by the drop and shaken (mol ratio CICOONB:triethylamine:DMAP 1:1.2:0.1). It is shaken for a further 15 minutes and the carrier then washed with approx. 200 ml of water-free acetone.

### b) Coupling of the Immunoglobulins to a Fixed Carrier

The polysaccharide matrix (gel) activated according to example 1a) is transferred to a watery medium by the step and then immediately stirred into the coupling solution, which contains the ligand. Citrate buffer pH 4.2 is used as a coupling buffer. Coupling is done under slight shaking for 2 h at room temperature. Free bindings are subsequently blocked by the addition of ethanolamine. Concrete conditions for the individual antibodies are stated in Tab. 1.

TABLE 1

| | | | | Ak | ml coupling | | |
|---|---|---|---|---|---|---|---|
| Gel | Chicken | | | solution | buffer (citrate, | Ethanolamine | moist |
| no. | Ak (IgY) | mg | mg/ml | (ml) | 0.1 M, pH 4.2) | 1 M (ml) | gel (g) |
| 1 | Cha. C3a | 8.3 | 9, 6, 7 | 0.6 | 4.4 | 0.5 | 5.77 |
| 2 | Cha. C5a | 10.3 | 10.2 | 1 | 4 | 0.5 | 5.63 |
| 3 | Cha. IL6 | 9.5 | 9.8 | 1 | 4 | 0.5 | 5.52 |
| 8 | Control | 0 | 0 | 0 | 5 | 0.5 | 5.60 |

### Example 2

The antibodies immobilised according to example 1 are used in order to remove interleukin 6 or complementary factors from liquid media such as buffer solutions, serum or blood plasma.

For this, the carriers are washed, transferred to a physiological buffer solution (PBS) and packed in plastic or glass columns free of bubbles. The arrangement is completed by connection to a chromatography device. The sample material to be adsorbed (buffer doted with the antigens, serum or blood plasma samples, doted or with natural antigen content) can now be guided across the immobilised antibodies specific for the antigens stated by gravity or with a suitable pump. The existing antigens are recognised by the IgY, firmly bound and thus removed from the medium flowing through the column. Effectivity is proven by analysis (ELISA) of the column throughflow, the antigen content of which has been reduced. After washing of the column with physiological buffer solution, the desorption of the bound antigen is done with suitable elution agents (0.1 M citrate buffer, pH 3), fractioning and analysis of the eluate. The quantitative proof of the antigens is used for determination of the capacity of the immuno-adsorbent.

| | | Coupling | Gel | Volume/ | | |
|---|---|---|---|---|---|---|
| | | density | volume | medium | Adsorbed | |
| Antigen | Experiment | [mg/ml gel] | [ml] | [ml] | quantity | |
| C3a | batch | 10 | 0.5 | 2/plasma | 12 | μg |
| | cyclic | 10 | 0.1 | 5/plasma | 2.6 | μg after 3 cycles |
| C5a | batch | 10 | 0.5 | 2/plasma | 4 | μg |
| | cyclic | 10 | 0.1 | 25/plasma | 2 | μg after 10 cycles |
| | | | | | 1.7 | μg after 3 cycles |
| IL6 | batch | 5 | 0.1 | 1/plasma | 133 | ng |
| | Affinity | 2 | 0.1 | 1/PBS + | 330 | ng |
| | chromatography | | | 1% BSA | | |
| | (1 cycle) | | | | | |
| | cyclic | 10 | 1 | 50/PBS + | 100 | ng |
| | | | | 1% BSA | | |
| | | | | | 94 | ng after 3 cycles |
| | | 5 | 1 | 50/PBS + | 93 | ng |
| | | | | 1% BSA | | |
| | | | | | 74 | ng after 3 cycles |

### Example 3

The antibodies immobilised according to example 1 are used in order to remove interleukin 6 and activated complement 5a from patients' blood plasma.

For this, the carriers are washed, transferred to a physiological buffer solution (PBS) and packed in a plastic housing free of bubbles. The adsorber modules contain 60 ml of gel. Two different adsorbers contain antibodies, each of which is aimed against C5a (10 mg IgY/ml sepharose gel [corresponds to an adsorption capacity of 400 μg/column] and IL6 [5 mg IgY/ml sepharose gel [corresponds to an adsorption capacity of 240 μg/column]). The modules are switched in series.

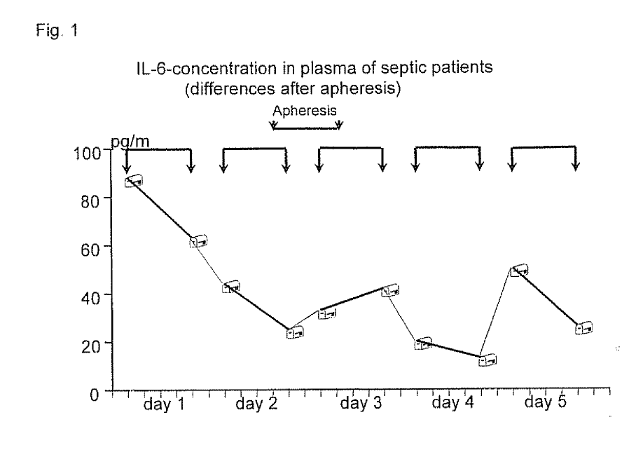

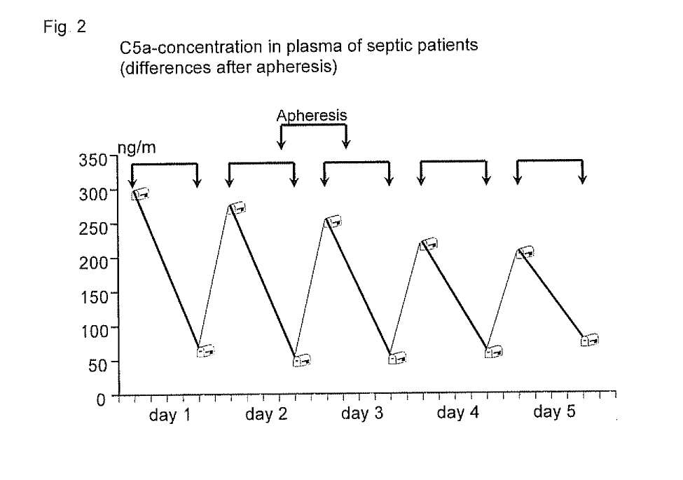

The following depletion kinetics were established on 5 consecutive days (5 litres of plasma treatment per day) (FIGS. 1 and 2):

1. Immunological adsorber for the treatment of inflammations,
wherein
there exist carrier materials of organic or synthetic polymers with bound poly- or monoclonal antibodies aimed against the complementary factors C3a and/or C5a and also, if applicable, with antibodies aimed against further inflammation mediators.

2. Immunological adsorbers according to claim 1, wherein the antibodies are polyclonal antibodies.

3. Immunological adsorbers according to claim 1, wherein the antibodies are aviary antibodies of type IgY.

4. Immunological adsorbers according to claims 1, wherein further antibodies against inflammation mediators are contained in accordance with the regulation condition of the inflammation reaction.

5. Immunological adsorbers according to claim 1, wherein these antibodies are aimed against TNF, IL1, IL6, IL8 and/or IL10.

6. Immunological adsorber according to claim 1, wherein C3a-specific antibodies and C5a-specific antibodies are contained.

7. Immunological adsorber according to claim 1, wherein antibodies aimed against C3a and/or C5a and TNF-specific antibodies are contained.

8. Immunological adsorber according to claim 1, wherein antibodies aimed against C3a and/or C5a and IL1-specific antibodies are contained.

9. Immunological adsorber according to claim 1, wherein antibodies aimed against C3a and/or C5a and IL6-specific antibodies are contained.

10. Immunological adsorber according to claim 1, wherein antibodies aimed against C3a and/or C5a and IL8-specific antibodies are contained.

11. Immunological adsorber according to claim 1, wherein antibodies aimed against C3a and/or C5a and IL10-specific antibodies are contained.

12. Immunological adsorber according to claim 1, wherein the bound antibodies are aimed against at least one of the following peptide sequences of the complementary factors C3a and C5a:

| | |
|---|---|
| C3a: | |
| NH₂-KCCEDGMRQNPMR-COOH | (SEQ ID NO 1) |
| NH₂-RFSCQRRTRFISL-COOH | (SEQ ID NO 2) |
| NH₂-ITELRRQHARAS-COOH | (SEQ ID NO 3 |
| C5a: | |
| NH₂-QADYKDDDDKLPAE-COOH | (SEQ ID NO 4) |
| NH₂-DDKLPAEGLDIENS-COOH | (SEQ ID NO 5) |

13. Immunological adsorber according to claim 1, wherein the bound antibodies are aimed against at least one of the following peptide sequences of interleukins 1α and 1β:

| | |
|---|---|
| IL1α: | |
| NH₂-NCYSENEEDSSSID-COOH | (SEQ ID NO 6) |
| NH₂-GAYKSSKDDAKIT-COOH | (SEQ ID NO 7) |
| NH₂-WETHGTKNYFTS-COOH | (SEQ ID NO 8 |
| IL1β: | |
| NH₂-RISDHHYSKGFRQA-COOH | (SEQ ID NO 9) |
| NH₂-VQGEESNDKIPVA-COOH | (SEQ ID NO 10) |
| NH₂-ESVDPKNYPKKKMEKRF-COOH | (SEQ ID NO 11) |

14. Immunological adsorber according to claim 1, wherein the bound antibodies are aimed against at least one of the following peptide sequences of interleukin 6:

| | |
|---|---|
| IL6: | |
| NH₂-APHRQPLTSSERIDKQI-COOH | (SEQ ID NO 12) |
| NH₂-QNRFESSEEQARA-COOH | (SEQ ID NO 13) |
| NH₂-AITTPDPTTNAS-COOH | (SEQ ID NO 14) |

15. Immunological adsorber according to claims 1, wherein the bound antibodies are aimed against at least one of the following peptide sequences of interleukin 10:

| | |
|---|---|
| IL10: | |
| NH₂-SPGQGTQSENSCT-COOH | (SEQ ID NO 15) |
| NH₂-QMKDQLDNLLLKES-COOH | (SEQ ID NO 16) |
| NH₂-MPQAENQDPDIKA-COOH | (SEQ ID NO 17) |
| NH₂-LPCENKSKAVEQ-COOH | (SEQ ID NO 18) |

16. Immunological adsorber according to claim 1, wherein the bound antibodies are aimed against at least one of the following peptide sequences:

| | |
|---|---|
| TNFα: | |
| NH₂-VRSSSRTPSDKPVA-COOH | (SEQ ID NO 19) |
| NH₂-KSPCQRETPEGAEAKPW-COOH | (SEQ ID NO 20) |

17. Immunological adsorber according to claim 1, wherein the organic or synthetic carrier material comprises membranes or particles of polystyrols, carbohydrates, such as cellulose or agarose derivatives, or acrylates.

18. Immunological adsorber according to claim 1, wherein the specific antibodies are covalently bound to the membranes or particles.

19. Immunological adsorber according to claim 1, wherein the antibodies are fixed to the carrier materials by spacers or linkers.

20. Method for the production of immunoadsorbers according to claims 1, wherein antibodies aimed against C3a and/or C5a and, if applicable, further sepsis mediators are covalently or adsorptively coupled to carrier materials of organic or synthetic polymers.

21. Method according to claim 1, wherein the antibodies are produced by immunisation preferably of small mammals, such as mice, rats or rabbits, or of birds, such as chickens, with the corresponding antigens.

22. Method according to claim 1, for the use of immunological adsorbers as an effective integral part of a device for the removal of complementary factors and, if applicable, further mediators from body fluids in a patient-specific combination.

23. Method according to claim 1, wherein the immunological adsorbers are used for plasma pheresis in patients with sepsis or septic shock and also other diseases connected with inflammations.
